(19) United States
(12) Patent Application Publication
(10) Pub. No.: US 2024/0221954 A1
(43) Pub. Date: Jul. 4, 2024

(54) DISEASE PREDICTION METHODS AND DEVICES, ELECTRONIC DEVICES, AND COMPUTER READABLE STORAGE MEDIA

(71) Applicants: Chengdu BOE Optoelectronics Technology Co., Ltd., Chengdu, Sichuan (CN); BOE TECHNOLOGY GROUP CO., LTD., Beijing (CN)

(72) Inventor: Mengjia LIU, Beijing (CN)

(21) Appl. No.: 17/922,017

(22) PCT Filed: Oct. 28, 2021
(86) PCT No.: PCT/CN2021/126970

§ 371 Date: Oct. 28, 2022

(51) Int. Cl. G16H 50/30; C12Q 1/6883

(52) U.S. Cl. CPC G16H 50/30; C12Q 1/6883

(57) ABSTRACT

The disclosure provides a disease prediction method, a disease prediction device, an electronic device and a computer readable storage medium. The disease prediction method comprises: acquiring gene sequencing data of a test sample; performing data analysis on the gene sequencing data to obtain a mutation site in the gene sequencing data; performing a mutation annotation of the mutation site to obtain mutation-related information of the mutation site; predicting a score of influence degree of the mutation site on gene function based on the mutation-related information of the mutation site; and annotating the mutation site with a disease based on the score of influence degree of the mutation site on gene function and a predefined disease database to obtain a mitochondrial disease corresponding to the mutation site.

### TECHNICAL FIELD

The present disclosure relates to the field of display technology, and specifically to disease prediction methods, disease prediction devices, electronic devices, and computer readable storage media.

### BACKGROUND

Gene mutation refers to a variation in bases (SNV) and a variation in the order of arrangement (indel) on genomic DNA molecules. The effects of the gene mutation on organisms vary greatly. The mutation in a non-gene region and a non-gene regulatory region has essentially no effect on the organism, while the mutation in a gene regulatory region can prevent normal transcription of genes. Mutation at gene exons, introns and the junction of exons may lead to mRNA degradation or affect normal translation of proteins, cause alteration of the three-dimensional structure of proteins, or incorrect subcellular localization of proteins, or affect normal transmembrane of proteins, enzyme activity, etc.

Mitochondria are organelles associated with energy metabolism and are integral to several life processes such as cell survival and cell death, where abnormal oxidative phosphorylation on the respiratory chain is associated with many human diseases. Common mitochondrial diseases include Leigh syndrome, deafness, encephalomyopathy, dystonia and the like. Mutation in these mitochondrial diseases involves point mutations, deletions, etc. The regions involved include mutations in rRNA/tRNA regions, and mutations in coding and non-coding regions.

### SUMMARY

The present disclosure provides a disease prediction method, a disease prediction device, an electronic device and a computer readable storage medium.

The present disclosure provides a disease prediction method, comprising:
acquiring gene sequencing data of a test sample;
performing data analysis on the gene sequencing data to obtain a mutation site in the gene sequencing data;
performing a mutation annotation of the mutation site to obtain mutation-related information of the mutation site;
predicting a score of influence degree of the mutation site on gene function based on the mutation-related information of the mutation site; and
annotating the mutation site with a disease based on the score of influence degree of the mutation site on gene function and a predefined disease database to obtain a mitochondrial disease corresponding to the mutation site,
wherein, when a first mitochondrial disease corresponding to the mutation-related information of the mutation site is recorded in the predefined disease database, the first mitochondrial disease is taken as the mitochondrial disease corresponding to the mutation site; when the first mitochondrial disease is not recorded in the predefined disease database and the score of influence degree is greater than a predetermined threshold, a second mitochondrial disease corresponding to an adjacent site of the mutation site is acquired from the predefined disease database and the second mitochondrial disease is taken as the mitochondrial disease corresponding to the mutation site.

In some embodiments, the mutation-related information comprises: a mutation type, a mutation region, a mutation location, and a mutation-induced variation in a CDS and a protein.

In some embodiments, predicting a score of influence degree of the mutation site on gene function based on the mutation-related information of the mutation site specifically comprises:
separately predicting the scores of influence degree of different mutation-related information on gene function to obtain multifaceted scores of influence degree, and
determining the score of influence degree of the mutation site on the gene function based on the multifaceted scores of influence degree.

In some embodiments, separately predicting the scores of influence degree of different mutation-related information on gene function to obtain the multifaceted scores of influence degree specifically comprises:
acquiring, as a first score, a score of influence degree of the mutation site on conservativeness and physicochemical properties of a protein;
acquiring, as a second score, a score of influence degree of the mutation type of the mutation site on gene function; and
acquiring, as a third score, a score of influence degree of the mutation location of the mutation site on gene function;
wherein the multifaceted scores of influence degree comprise: the first score, the second score and the third score.

Determining the score of influence degree of the mutation site on the gene function based on the multifaceted scores of influence degree specifically comprises: determining the score of influence degree of the mutation site on the gene function according to the following formula,

$$S = λ1 * Si + λ2 * St + λ3 * Sp ,$$

wherein S is the score of influence degree of the mutation site on the gene function, Si is the first score, St is the second score; Sp is the third score; λ1, λ2 and λ3 are predetermined weights, and λ1+λ2+λ3=1.

In some embodiments, λ1 and λ2 each range from 0.15 to 0.25, and λ3 ranges from 0.5 to 0.7.

In some embodiments, acquiring the score of influence degree of the mutation site on the conservativeness and physicochemical properties of the protein specifically comprises:
analyzing the mutation-related information of the mutation site separately by a plurality of prediction tools to predict a plurality of reference scores of influence degree of the mutation site on the conservativeness and physicochemical properties of the protein, and
averaging the plurality of reference scores of influence degree as the first score.

In some embodiments, acquiring the score of influence degree of the mutation type of the mutation site on the gene function comprises:
determining, based on a predetermined first mapping relationship, the score of influence degree of the mutation type of the mutation site on the gene function; wherein scores of influence degree of a plurality of different mutation types on gene function are recorded in the first mapping relationship.

In some embodiments, the mutation location comprises a location number n of the mutation site in a protein sequence.

Acquiring the score of influence degree of the mutation location of the mutation site on gene function specifically comprises:
determining the third score according to the following formula, when the mutation type of the mutation site is a drift mutation or a nonsense mutation,
Sp=1−n/L, where L is the length of the protein sequence, or
determining the third score as 0 when the mutation type of the mutation site is any other than the drift mutation and the nonsense mutation.

In some embodiments, acquiring the gene sequencing data of a test sample comprises:
acquiring initial gene sequencing data of the test sample, and
filtering the initial gene sequencing data to obtain the gene sequencing data.

In some embodiments, acquiring the initial gene sequencing data of the test sample comprises:
acquiring the initial gene sequencing data of the test sample using Nanopore sequencing technology or targeted enrichment sequencing technology.

In some embodiments, performing data analysis on the gene sequencing data to obtain the mutation site in the gene sequencing data specifically comprises:
comparing the gene sequencing data with a reference sequence of a reference mitochondrial genome, and determining comparison result data, the comparison result data comprising: a site of the gene sequencing data in the reference mitochondrial genome, and
performing a mutation detection on the comparison result data to determine the mutation site in the comparison result data.

In some embodiments, performing a mutation detection on the comparison result data to determine the mutation site in the comparison result data specifically comprises:
performing a SNV detection on the comparison result data to obtain a first detection result, the first detection result comprising: a SNV site included in the comparison result data; and
performing an indel detection on the comparison result data to obtain a second detection result, the second detection result comprising: an indel site included in the comparison result data;
wherein the mutation site comprises the SNV site and the indel site.

Some embodiments of the present disclosure provide a disease prediction device, comprising:
a data acquisition module configured to acquire gene sequencing data of a test sample;
an analysis module configured to perform data analysis on the gene sequencing data to obtain the mutation site in the gene sequencing data;
a mutation annotation module configured to perform mutation annotation of the mutation site to obtain mutation-related information of the mutation site;
a prediction module configured to predict a score of influence degree of the mutation site on gene function based on the mutation-related information of the mutation site; and
a disease annotation module configured to annotate the mutation site with a disease based on the score of influence degree of the mutation site on gene function and a predefined disease database to obtain a mitochondrial disease corresponding to the mutation site;
wherein, when a first mitochondrial disease corresponding to the mutation-related information of the mutation site is recorded in the predefined disease database, the first mitochondrial disease is taken as the mitochondrial disease corresponding to the mutation site; when the first mitochondrial disease is not recorded in the predefined disease database and the score of influence degree is greater than a predetermined threshold, a second mitochondrial disease corresponding to an adjacent site of the mutation site is obtained from the predefined disease database and the second mitochondrial disease is taken as the mitochondrial disease corresponding to the mutation site.

Some embodiments of the present disclosure provide an electronic device comprising a memory and a processor, the memory having a computer program stored thereon, wherein the computer program, when executed by the processor, implements the above method.

Some embodiments of the present disclosure provide a computer readable storage medium having a computer program stored thereon, wherein the computer program, when executed by a processor, implements the above method.

### BRIEF DESCRIPTION OF DRAWINGS

The accompanying drawings are intended to provide a further understanding of the present disclosure and form part of the specification, and they are used in conjunction with the specific embodiments below to explain the present disclosure, but do not constitute a limitation of the present disclosure. In the accompanying drawings:

FIG. 1 is a schematic diagram of a disease prediction method provided in some embodiments of the present disclosure.

FIG. 2 is a schematic diagram of a disease prediction method provided in some other embodiments of the present disclosure.

FIG. 3 is a statistical graph of the read length distribution of the raw data provided in an example of the present disclosure.

FIG. 4A is a composition diagram of the first 100 bases in the raw data reads provided in an example of the present disclosure.

FIG. 4B is a composition diagram of the last 100 bases in the raw data reads provided in an example of the present disclosure.

FIG. 5A is a graph of the mean base quality of the first 100 bases of the raw data provided in an example of the present disclosure.

FIG. 5B is a graph of the mean base quality of the last 100 bases of the raw data provided in an example of the present disclosure.

FIG. 6 is a schematic diagram of the visual output result of the statistics of comparison result data from step S2a provided in an example of the present disclosure.

FIG. 7 is a schematic diagram of an optional implementation of the step S4 provided in some embodiments of the present disclosure.

FIG. 8 is a schematic diagram of a disease prediction device provided in some embodiments of the present disclosure.

FIG. 9 is a schematic diagram of an electronic device provided in some embodiments of the present disclosure.

FIG. 10 is a schematic diagram of a computer readable storage medium provided in some embodiments of the present disclosure.

### DETAILED DESCRIPTION OF EMBODIMENTS

Some specific embodiments of the present disclosure will be described in detail below with reference to the accompanying drawings. It should be understood that the specific embodiments described herein are intended to illustrate and explain the present disclosure only and are not intended to limit the present disclosure.

Some terminology used in specific embodiments of the present disclosure is explained as follows.

High-throughput sequencing, also known as “next generation” sequencing (NGS), is characterized by the ability to sequence hundreds of thousands to millions of DNA molecules in parallel at a time and generally by shorter read lengths. Sequencing refers to the analysis of the base sequence of a specific DNA fragment, i.e., the arrangement of adenine (A), thymine (T), cytosine (C) and guanine (G). The advent of rapid DNA sequencing methods has greatly advanced research and discovery in biology and medicine.

Read: the sequence obtained after high-throughput sequencing, containing the sequenced base information, and quality value information.

Nanopore: the nanopore single-molecule sequencing technology, using electrical signals and nucleic acid endonucleases for sequencing, with very long sequencing length (usually the average length can range from a dozen Kbp to tens of Kbp).

Genome: in the field of molecular biology and genetics, the genome is the sum of all the genetic materials of an organism. These genetic materials include DNA or RNA. The genome includes coding and non-coding DNA, mitochondrial DNA and chloroplast DNA.

Gene mutation: in biological terms, it means a variation in the genetic genes (usually deoxyribonucleic acid present in the nucleus) of a cell. It includes point mutations caused by a variation in a single base, or deletions, duplications and insertions of multiple bases. The cause may be derived from errors in the replication of genetic genes during cell division, or from the effects of chemicals, genotoxicity, radiation, or viruses.

SNV (single nucleotide variation): a variation in a single DNA base.

Indel (insertion or deletion): a type of mutation that is an insertion or deletion in DNA.

hgvs (human genome variation society): the standard format for human genome variation.

The traditional method of identifying a mitochondrial disease is done mainly through clinical biochemistry, but there are problems such as high requirements for physicians, possible misclassification and omission, and difficulties in determining relatively rare types of mitochondrial diseases, as well as disadvantages such as low throughput, complicated operation and long cycle time. Usually, the methods for detecting mitochondrial diseases by gene sequencing only have the ability to identify known mutations, but do not have the ability to identify mutations that have not been reported and that also affect genes and protein functions (i.e., the methods cannot detect by which disease the mutation is caused).

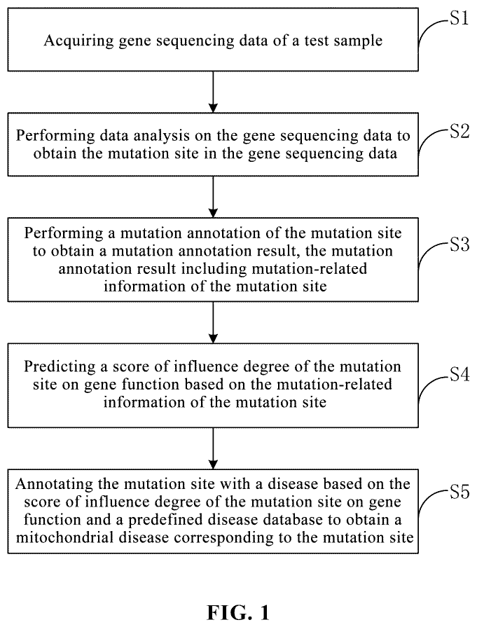

FIG. 1 is a schematic diagram of a disease prediction method provided in some embodiments of the present disclosure, which is particularly suitable for predicting a mitochondrial disease by genetic testing. As shown in FIG. 1, the disease prediction method comprises:

S1: acquiring gene sequencing data of a test sample.

Among others, the test sample may be a DNA sample from a patient with a mitochondrial disease, for example, a plasma or a serum from a patient. The gene sequencing data of the test sample can be obtained by sequencing with a third-generation sequencer.

S2: performing data analysis on the gene sequencing data to obtain the mutation site in the gene sequencing data.

Mutation sites are the base types that are different from the reference genome at the same positions in the gene sequencing data set of the test sample, and these mutation sites may be the pathogenic sites that affect human health, or cause disease in humans.

In some embodiments, performing data analysis of the gene sequencing data in step S2 may include: quality control and filtering of the gene sequencing data to obtain high quality data, and performing a gene detection based on the filtered data to identify the mutation site. There can be various types of gene detection. For example, the sites where SNV mutations occur can be detected by means of SNV detection.

S3: performing a mutation annotation of the mutation site to obtain a mutation annotation result, the mutation annotation result including mutation-related information of the mutation site.

In some embodiments, the mutation-related information may include: the type of mutation, the location of mutation, and the alteration of CDS bases and proteins due to the mutation.

S4: predicting a score of influence degree of the mutation site on gene function based on the mutation-related information of the mutation site.

The score of influence degree is used to indicate the degree of influence of the mutation site on the gene function. The higher the score of influence degree, the higher the impact of the mutation site on gene function; and the lower the score of influence degree, the lower the impact of the mutation site on gene function.

The influence degree on gene function may vary depending on the mutation type of the mutation site, and the influence degree on gene function may also vary depending on the mutation location of the mutation site. Therefore, the score of influence degree of the mutation site on gene function can be predicated based on the mutation-related information of the mutation site.

S5: annotating the mutation site with a disease based on the score of influence degree of the mutation site on gene function and a predefined disease database to obtain a mitochondrial disease corresponding to the mutation site.

Among others, the predefined disease database has recorded the correspondence between known mutation-related information and mitochondrial diseases. That is, for the mutation-related information of certain mutation sites, the corresponding mitochondrial diseases can be determined by looking up the predefined disease database. However, there are cases where the disease directly corresponding to the mutation-related information of the mutation site cannot be found from the predefined disease database, and then the mitochondrial disease corresponding to the mutation site can be determined based on the score of influence degree.

For example, when the first mitochondrial disease corresponding to the mutation-related information of the mutation site is recorded in the predefined disease database, the first mitochondrial disease is taken as the mitochondrial disease corresponding to the mutation site. When the first mitochondrial disease corresponding to the mutation-related information of the mutation site is not recorded in the predefined disease database and the score of influence degree calculated in step S4 is greater than a predefined threshold, a second mitochondrial disease corresponding to an adjacent site of the mutation site is obtained from the predefined disease database as the mitochondrial disease corresponding to the mutation site. When the first mitochondrial disease corresponding to the mutation-related information of the mutation site is not recorded in the predefined disease database and the score of influence degree obtained from step S4 is not greater than the predefined threshold, the mutation site is considered to have little impact on protein function and is not sufficient to cause disease.

Among others, the adjacent site is defined as the site closest to the mutation site among all the sites that satisfy the following two conditions. The first condition is that the mitochondrial disease corresponding to the mutation-related information thereof is recorded in the predefined disease database; and the second condition is that the site is located on the same gene and the same protein as the mutation site. For example, it is determined in step S2 that the mutation site is the second site on a protein sequence, and it is determined in step S4 that the score of influence degree of the mutation site on gene function is greater than a predetermined threshold, and no mitochondrial disease directly corresponding to the mutation-related information of the mutation site is recorded in the predefined disease database, but the mitochondrial diseases corresponding to the fourth site and the tenth site on the same protein are recorded. In this case, the mitochondrial disease corresponding to the fourth site is taken as the mitochondrial disease corresponding to the mutation site.

In some examples, the predetermined threshold may range from 0.4 to 0.5, for example, a predetermined threshold of 0.4, or 0.45, or 0.5.

In embodiments of the present disclosure, after identifying the mutation site in the gene sequencing data, mutation annotation is performed on the mutation site to obtain the mutation-related information of the mutation site, and then the score of influence degree of the mutation site on gene function is predicated based on the mutation-related information. Based on the score of influence degree and the predefined disease database, a disease annotation is performed on the mutation site, such that when a first mitochondrial disease directly corresponding to the mutation-related information of the mutation site is recorded in the predefined disease database, the first mitochondrial disease is taken as the mitochondrial disease corresponding to the mutation site; and when the first mitochondrial disease directly corresponding to the mutation-related information of the mutation site is not recorded in the predefined disease database, and the score of influence degree is greater than a predefined threshold, the second mitochondrial disease corresponding to the adjacent site of the mutation site recorded in the predefined disease database is taken as the mitochondrial disease corresponding to the mutation site. Thus, embodiments of the present disclosure can identify mitochondrial diseases not only when known mutation occurs in the genome, but also when unknown mutation occurs in the genome.

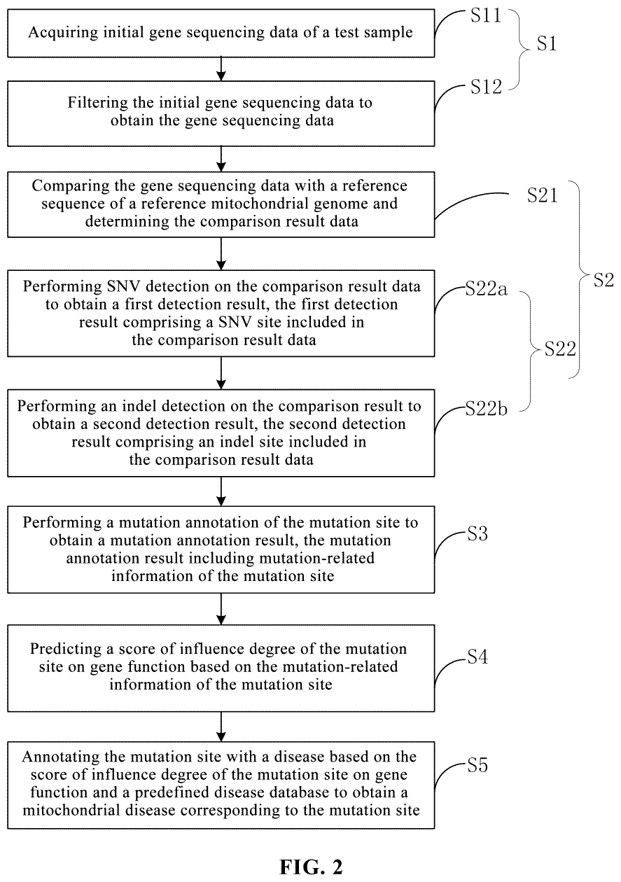

FIG. 2 is a schematic diagram of the disease prediction method provided in some other embodiments of the present disclosure, and FIG. 2 shows a particularized implementation of FIG. 1. As shown in FIG. 2, the disease prediction method comprises:

S1: acquiring gene sequencing data of a test sample.

In some embodiments, step S1 comprises step S11 and step S12:

S11: acquiring initial gene sequencing data of the test sample.

In some embodiments, in step S11, the test sample can be genetically sequenced using Nanopore sequencing technology or targeted enrichment sequencing technology to obtain the initial gene sequencing data.

For example, the gene sequencing may be performed using Nanopore sequencing technology. Compared with NGS sequencing, the Nanopore sequencing technology has a longer read length and has unparalleled advantages in plant and animal genome assembly.

S12: filtering the initial gene sequencing data to obtain the gene sequencing data.

In some embodiments, the step S12 may specifically include: analyzing, quality controlling and filtering the initial gene sequencing data to obtain high quality data for subsequent bioinformatic analysis and providing accurate data for subsequent analytical processing.

In one example, the nanopack analysis software package is used for analysis, quality control and filtering. NanoQC software is used for statistics of nucleic acid composition and base quality; NanoStat software is used to supplement the statistics and generate html files of statistical results; and NanoPlot software is used for visual graphing of the data. Subsequently, NanoFit software is used for filtering, and some sequences with too short lengths can be filtered out in the filtering process.

Among them, the filtering parameters are designed according to actual situations. For example, the filtering parameter is “-Q 7 -l 1000 -headcrop 100 -tailcrop 100”, meaning that the sequences having a length less than 1000 and an average quality value of the whole sequence less than Q7 are filtered out, while each sequence is cleaved for 100 bp at the head and the tail. In an example, the specific filtering information is shown in Table 1.

TABLE 1

| | Prior to filtration | After filtration |
|---|---|---|
| Mean read length | 1921.5 | 2490.2 |
| Mean read quality | 9.9 | 10.2 |
| Median read length | 1442.5 | 1686 |
| Median read quality | 10.1 | 10.3 |
| Number of reads | 110192 | 65246 |
| Read length N50 | 2336 | 2870 |
| STDEV read length | 2132.9 | 2457.6 |
| Total bases | 211728431 | 162473151 |
| Q5 | 110184 (100.0%) 211.7 Mb | 65246 (100.0%)162.5 Mb |
| Q7 | 110080 (99.9%) 211.3 Mb | 65245(100.0%)162.5 Mb |
| Q10 | 61612 (55.9%) 100.5 Mb | 35441(54.3%) 72.8 Mb |
| Q12 | 18846 (17.1%) 32.3 Mb | 13382(20.5%)27.1 Mb |
| Q15 | 6 (0.0%) 0.0 Mb | 16 (0.0%) 0.0 Mb |

In Table 1, Mean read length: the length of mean read; Mean read quality: the quality of mean read; Median read length: the median of read length; Median read quality: the median of read quality; Number of reads: total number of reads; Read length N50: read length of N50 value; STDEV read length: standard variance of read length; Q5-Q15: statistics of quality values of Nanopore, including the number of reads, the percentage thereof relative to total number, and the total number of bases.

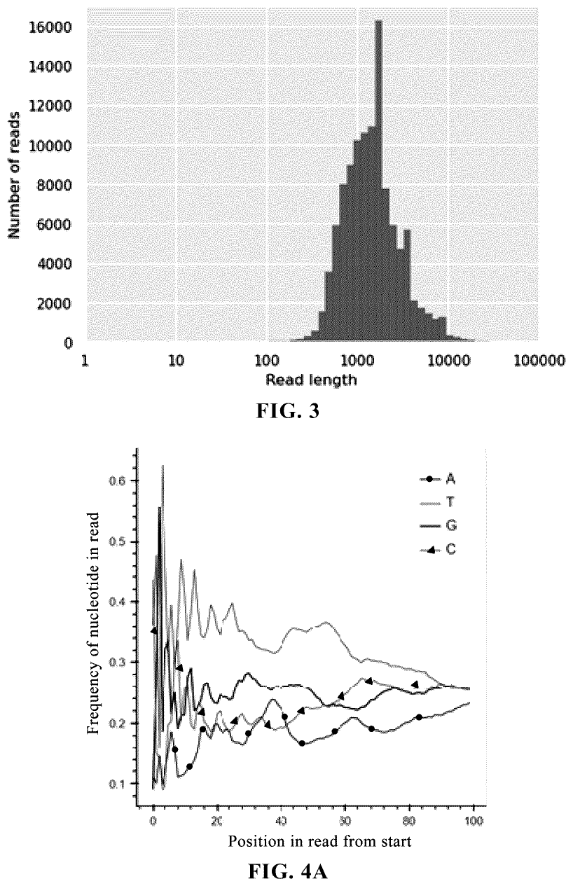

FIG. 3 is a statistical graph of the read length distribution of the raw data (i.e., initial gene sequencing data) in an example of the present disclosure, where the horizontal axis represents the read length and the vertical axis represents the number of reads. As shown in FIG. 3, the read lengths are generally distributed around 1000.

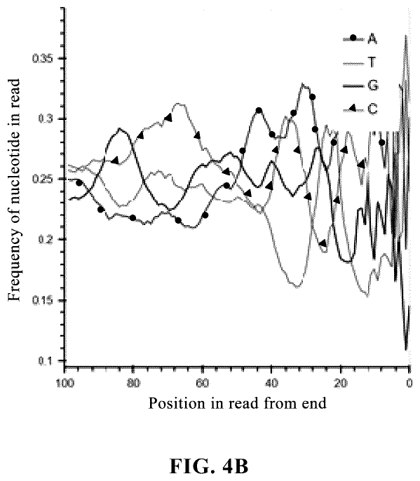

FIG. 4A is a composition diagram of the first 100 bases in the raw data reads provided in an example of the present disclosure, and FIG. 4B is a composition diagram of the last 100 bases in the raw data reads provided in an example of the present disclosure. In particular, the vertical axis of FIG. 4A and FIG. 4B indicates the frequency of nucleotide in read, the horizontal axis in FIG. 4A indicates the position in the sequence from the head (position in read from start), and the horizontal axis in FIG. 4B indicates the position in the sequence from the tail (position in read from end).

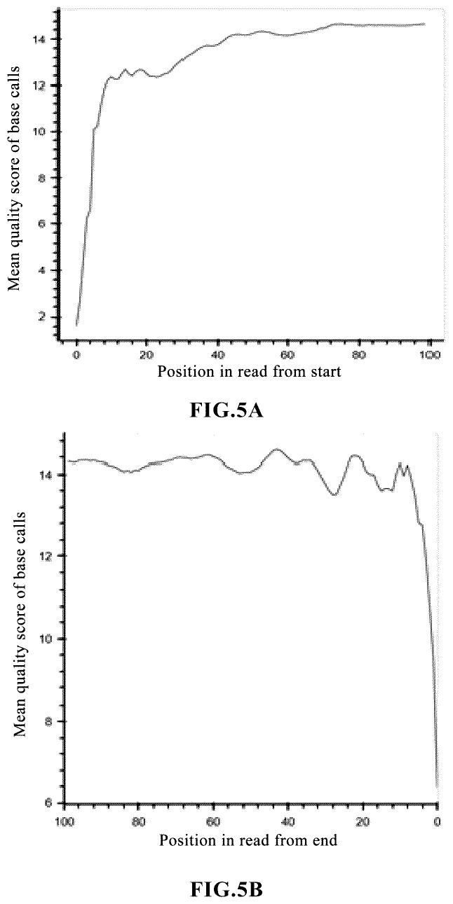

FIG. 5A is a graph of the mean base quality of the first 100 bases in the raw data provided in an example of the present disclosure, and FIG. 5B is a graph of the mean base quality of the last 100 bases in the raw data provided in an example of the present disclosure. In particular, the vertical axis in FIG. 5A and FIG. 5B indicates the average base quality (mean quality score of base calls), the horizontal axis in FIG. 5A indicates the position in the sequence from the head (position in read from start), and FIG. 5B indicates the position in the sequence from the tail (position in read from end).

S2: performing data analysis on the gene sequencing data to obtain the mutation site in the gene sequencing data.

In some embodiments, step S2 comprises step S21 and step S22:

S21: comparing the gene sequencing data with a reference sequence of a reference mitochondrial genome and determining the comparison result data, the comparison result data comprising: the site in the mitochondrial genome in the gene sequencing data.

By step S21, the site in the mitochondrial genome of the gene sequencing data can be determined.

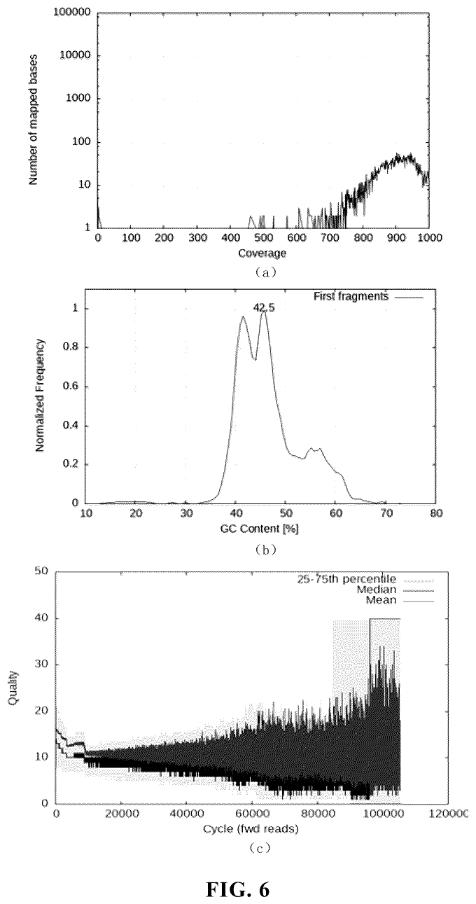

In one example, the comparison may be performed using the minimap2 tool with the comparison parameter “-ax map-out” and the generated results of the minimap2 tool are present in sam format. The comparison data in sam format is converted into bam format and sorted by the samtools. Then, comparison statistics are performed using the flagstat and stats commands in the samtools, and the comparison results are visualized and output using the plot-bamstats program in the samtools. FIG. 6 is a schematic diagram of the visual output result of the statistics of comparison result data from step S2a provided in an example of the present disclosure. FIG. 6(a) shows the coverage graph obtained from statistic of the comparison result data, wherein the horizontal axis indicates the coverage, and the vertical axis indicates the number of bases that can be mapped to the reference mitochondrial genome (Number of mapped bases). FIG. 6(b) shows the GC distribution from statistic of the comparison results, with the horizontal axis indicating the GC content and the vertical axis indicating the normalized frequency. FIG. 6(c) shows the statistical graph of the quality distribution of the reads that can be mapped to the reference mitochondrial genome, with the horizontal axis indicating the length of the reads that can be mapped to the reference mitochondrial genome, i.e., Cycle (fwd reads), and the vertical axis indicating the quality value (Quality).

S22: performing mutation detection on the comparison result data to determine the mutation site in the comparison result data.

In some embodiments, step S22 specifically comprises:

S22a: performing SNV detection on the comparison result data to obtain a first detection result, the first detection result comprising: a SNV site included in the comparison result data.

S22b: performing an indel detection on the comparison result to obtain a second detection result, the second detection result comprising: an indel site included in the comparison result data.

Exemplarily, SNV detection may be performed using the longshot tool, which is an excellent mutation detection tool for accurate detection of read length data with errors, and which uses a bam file as input and outputs a vcf file with mutation information and genotype information. The indel on the mitochondria may be detected using mitoDel V3.0, and the output result of the indel includes the number of read count from the indel, the starting position of the indel, the location of the indel and whether it passes the quality filtering.

It should be understood that the mutation site detected in the step S2 above includes the SNV site and the indel site.

It should be noted that the sequence of step S22a and step S22b is not particularly limited, as long as the SNV detection and indel detection are performed separately. By performing the SNV detection and indel detection separately, the accuracy of the detection can be improved.

S3: performing a mutation annotation of the mutation site to obtain mutation-related information of the mutation site.

In some embodiments, the mutation-related information may specifically include: mutation type, mutation region, mutation location; and mutation-induced variation in the CDS and protein of cysteine sulfinate decarboxylase, with CDS being the sequence encoding the protein on the gene. Among them, the mutation type may be nonsense mutation, drift mutation, synonymous mutation, etc., and the mutation region may be non-genetic region, genetic region, control region, etc. The mutation location refers to the specific position of the mutation site located in the genome that can be mapped to the position on a specific gene and protein sequence, indicating at which position the specific transcript of the gene, CDS or the protein is altered.

S4: predicting the score of influence degree of the mutation site on gene function based on the mutation-related information.

In some embodiments, step S4 comprises: separately predicting the scores of influence degree of different mutation-related information on gene function to obtain multifaceted scores of influence degree; and determining the score of influence degree of the mutation site on the gene function based on the multifaceted scores of influence degree. For example, the multifaceted scores of influence degree may be obtained by separately predicating the scores of influence degree of the mutation type, mutation location, and other information on gene function.

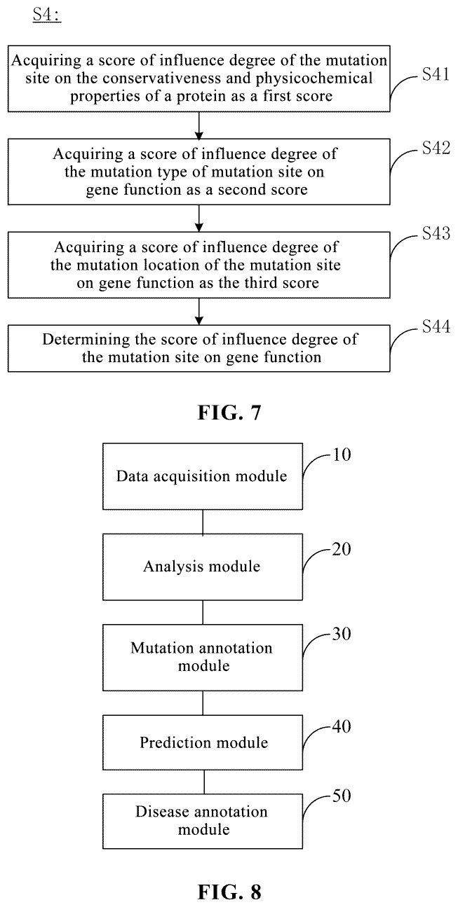

FIG. 7 is a schematic diagram of an optional implementation of step S4 provided in some embodiments of the present disclosure. In some embodiments, step S4 specifically comprises step S41 to step S44.

S41: acquiring a score of influence degree of the mutation site on the conservativeness and physicochemical properties of a protein, as a first score.

Among others, the step S41 may specifically include step S41a and step S41b.

S41a: analyzing the mutation-related information of the mutation site separately by multiple prediction tools to predict a plurality of reference scores of influence degree of the mutation site on conservativeness and physicochemical properties of the protein.

S41b: averaging the plurality of reference scores of influence degree as the first score.

For example, the multiple prediction tools include: PANTHER, PolyPhen-2 and SIFT, each predicting the influence degree of the mutation site on conservativeness and physicochemical properties of the protein, wherein the influence degree of the mutation site on conservativeness and physicochemical properties of the protein may be one of the following four outcomes: no influence, possible influence, deleterious, and unable to predict. When the mutation site has no influence on the conservativeness and physicochemical properties of the protein, the reference score of influence degree output by the prediction tool is 0; when the mutation site has possible influence on the conservativeness and physicochemical properties of the protein, the reference score of influence degree output by the prediction tool is 0.5; when the mutation site has a deleterious influence on the conservativeness and physicochemical properties of the protein, the reference score of influence degree output by the prediction tool is 1. When the influence degree of the mutation site on the conservativeness and physicochemical properties of the protein is not predictable, the reference score of influence degree output by the prediction tool is NA (no score).

The reference score of influence degree obtained from PANTHER is denoted as S_{PANTHER}, the reference score of influence degree obtained from PolyPhen-2 is denoted as S_{PolyPhen-2}, and the reference score of influence degree obtained from SIFT is denoted as S_{SIFT}. Then, the first score Si=(S_{PANTHER}+S_{PolyPhen-2}+S_{SIFT})/N, wherein N is the number of prediction tools giving scores, i.e., the number of prediction tools exclusive of NA.

S42: acquiring a score of influence degree of the mutation type of the mutation site on the gene function, as a second score.

In some embodiments, the step S42 may specifically include: determining the score of influence degree of the mutation type of the mutation site on gene function based on a predetermined first mapping relationship; wherein the first mapping relationship has recorded scores of influence degree of a plurality of different mutation types on gene function.

For example, the same mutation and intergenic region mutation usually have no influence on gene function; therefore, the second score is 0 when the mutation type of the mutation site is the same mutation or intergenic region mutation. Nonsynonymous mutation and non-drift mutation have possible influence on protein function; therefore, the second score is 0.5 when the mutation type of the mutation site is nonsynonymous mutation or non-drift mutation. Nonsense mutation and drift mutation have great influence on protein function; therefore, the second score is 1 when the mutation type of the mutation site is nonsense mutation or drift mutation.

S43: acquiring a score of influence degree of the mutation location of the mutation site on the gene function, as a third score.

In some embodiments, the mutation location may include: the location number n of the mutation site in the protein sequence, meaning that the mutation site is located at the position of the n^{th} amino acid in the protein sequence.

S43 may specifically include the following. When the mutation type of the mutation site is a drift mutation or a nonsense mutation, the third score Sp=1−n/L, where L is the length of the protein sequence. When the mutation type of the mutation site is any other than drift mutation and nonsense mutation, the third score is determined as 0. For example, if a protein sequence has a length of 200 amino acids, and the mutation site is at the 20^{th} amino acid position of the protein sequence, and the mutation type is drift mutation or nonsense mutation, then Sp=1−20/200=0.9.

S44: determining the score S of influence degree of the mutation site on gene function according to the following equation (1):

$$S = λ1 * Si + λ2 * St + λ3 * Sp$$

where Si is the first score; St is the second score; Sp is the third score; λ1, λ2, and λ3 are predetermined weights, and λ1+λ2+λ3=1. Larger S means greater influence of the mutation site on the protein. When S=0, it means no influence on the protein function at all. When S=1, it means harmful to the protein, and the protein cannot exercise its function at all.

Among others, the above-mentioned “separately predicting the scores of influence degree of different mutation-related information on gene function to obtain multifaceted scores of influence degree” includes the above steps S41 to S43. The multifaceted scores of influence degree include: the first score, the second score and the third score. The above-mentioned “determining the score of influence degree of the mutation site on the gene function based on the multifaceted scores of influence degree” includes the above step S44.

S5: performing disease annotation of the mutation site based on the score of influence degree of the mutation site on the gene function and the predefined disease database to obtain the mitochondrial disease corresponding to the mutation site. Among others, when a first mitochondrial disease corresponding to the mutation-related information of the mutation site is recorded in the predefined disease database, the first mitochondrial disease is taken as the mitochondrial disease corresponding to the mutation site; when the first mitochondrial disease is not recorded in the predefined disease database and the score of influence degree is greater than a predefined threshold, a second mitochondrial disease corresponding to the adjacent site of the mutation site is obtained from the predefined disease database, and the second mitochondrial disease is taken as the mitochondrial disease corresponding to the mutation site.

In some examples, when performing step S5, the mutation disease data on the mitomap may be downloaded to construct a database in tsv format (referred to as “mitoDisease”), which serves as the predefined disease database. In some examples, the predetermined threshold may be 0.5.

Some of the mitochondrial diseases with the corresponding mutation-related information are shown in Table 2.

TABLE 2

| | | |
|---|---|---|
| Mutation Num. | 19 | 32 |
| Position | 9025 | 15672 |
| Mutation(hgvs.g) | m.9025G > A | m.15672T > G |
| Mutation(hgvs.c) | c.499G > A | c.926T > G |
| Mutation(hgvs.p) | p.G167S | p.I309R |
| S | 0.549 | 0.41 |
| IF | 1 | 0 |
| (mitoDisease) | | |
| mitoDisease Num. | 242 | 475 |
| mitoDisease-gene | ATP6 | CYB |
| Disease | Motor neuropathy, | LHON |
| | Leigh-like, colon cancer | |
| Status | Reported | — |
| GB Freq | 0.06% | — |

In Table 2, “Mutation Num” indicates the mutation annotation number; “Position” indicates the location of the mutation site on the mitochondrial genome; “Mutation(hgvs.g)”, “Mutation(hgvs.c)”, and “Mutation(hgvs.p)” indicate the standard hgvs formats of genome, CDS, and protein, respectively; “S” indicates the score of influence degree of the mutation site on gene function; “IF (mitoDisease)” indicates whether the current mutation is present in the mitoDisease database, and particularly, “1” indicates that the current mutation exists in the database, while “0” indicates that the current mutation does not exist in the database. If the current mutation exists in the database, “mitoDisease Num” indicates the number of the current mutation in the mitoDisease database; or if the current mutation does not exist in the database, “mitoDisease Num” indicates the number of the mutation at the adjacent site of the current mutation site in the mitoDisease database. In addition, “mitoDisease-gene” indicates the name of the gene to which the current mutation corresponds in the mitoDisease. “Disease” indicates the name of the disease associated with the mutation. “Status” indicates whether the current mutation has been reported, and “Reported” means that the current mutation has been reported. “GB Freq” indicates the frequency of the current mutation in the human mitochondrial database in GeneBank.

FIG. 8 is a schematic diagram of a disease prediction device provided in some embodiments of the present disclosure, which is used to perform the disease prediction methods described above. As shown in FIG. 8, the disease prediction device includes: a data acquisition module 10, an analysis module 20, a mutation annotation module 30, a prediction module 40, and a disease annotation module 50.

Among them, the data acquisition module 10 is configured to acquire gene sequencing data of the test sample.

The analysis module 20 is configured to perform data analysis on the gene sequencing data to obtain a mutation site in the gene sequencing data.

The mutation annotation module 30 is configured to perform mutation annotation of the mutation site to obtain the mutation-related information of the mutation site.

The prediction module 40 is configured to predict the score of influence degree of the mutation site on gene function based on the mutation-related information of the mutation site.

The disease annotation module 50 is configured to annotate the mutation site with a disease based on the score of influence degree of the mutation site on gene function and a predefined disease database to obtain a mitochondrial disease corresponding to the mutation site. Among others, when a first mitochondrial disease corresponding to the mutation-related information of the mutation site is recorded in the predefined disease database, the first mitochondrial disease is taken as the mitochondrial disease corresponding to the mutation site; when the first mitochondrial disease is not recorded in the predefined disease database and the score of influence degree is greater than a predefined threshold, a second mitochondrial disease corresponding to the adjacent site of the mutation site is acquired from the predefined disease database, and the second mitochondrial disease is taken as the mitochondrial disease corresponding to the mutation site.

Among them, the functions of the respective modules are described in the above disease prediction methods and will not be repeated here.

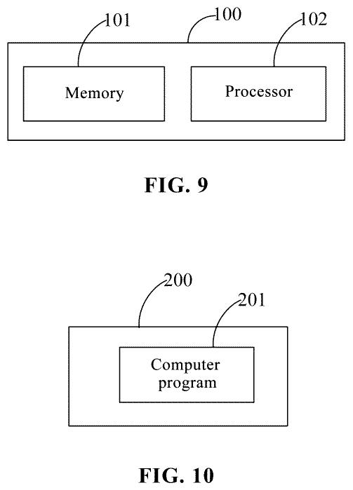

FIG. 9 is a schematic diagram of an electronic device provided in some embodiments of the present disclosure. As shown in FIG. 9, the electronic device 100 comprises: a memory 101 and a processor 102, wherein a computer program is stored on the memory 101, wherein the computer program, when executed by the processor 102, implements the disease prediction methods described above, such as steps S1 to S4 in FIG. 1.

The electronic device 100 may be a computing device such as a desktop computer, a laptop computer, a handheld computer, and a cloud-based server. The electronic device 100 may include, but is not limited to, a processor 102 and a memory 101. It will be understood by those of skill in the art that FIG. 9 is merely an example of the electronic device 100 and does not constitute a limitation of the electronic device 100, which may include more or fewer components than shown, or a combination of certain components, or different components. For example, the electronic device 100 may also include input and output devices, network access devices, buses, etc.

The processor 102 may be a central processing unit (CPU), but may also be other general purpose processors, digital signal processors (DSP), application specific integrated circuits (ASIC), field-programmable gate arrays (FPGA) or other programmable logic devices, discrete gate or transistor logic devices, discrete hardware components, etc. The general purpose processor 102 may be a microprocessor, or the processor may also be any conventional processor, etc.

The memory 101 may be an internal storage unit of the electronic device 100, such as a hard disk or memory of the electronic device 100. The memory 101 may also be an external storage device of the electronic device 100, such as a plug-in hard disk, smart media card (SMC), secure digital (SD) card, flash card, etc., equipped on the electronic device 100. Further, the memory 101 may also include both internal storage units and external storage devices of the electronic device 100. The memory 101 is used to store the computer program and other programs and data required by the terminal device. The memory 101 may also be used to temporarily store data that has been output or will be output.

It will be obvious to those skilled in the art that, for the sake of convenience and brevity, description is made with reference to the above configuration of functional units and modules as an example. In practice, the above-mentioned functions can be assigned to different functional units and modules as needed, or in other words, the internal structure of the device is divided into different functional units or modules to perform all or some of the above-mentioned functions. Each functional unit and module in the embodiments can be integrated in a processing unit, or each unit may physically exist separately, or two or more units can be integrated in a single unit, and the above integrated unit may be implemented either in the form of hardware or by software functional units. In addition, the specific names of the functional units and modules are only for the purpose of mutual distinction and are not used to limit the scope of protection of the present disclosure. As for the specific working processes of the units and modules in the above systems, reference may be made to the corresponding processes described above in the embodiments of the methods, and will not be repeated here.

FIG. 10 is a schematic diagram of a computer readable storage medium provided in some embodiments of the present disclosure. As shown in FIG. 10, a computer program 201 is stored on the computer readable storage medium 200, wherein the computer program 201, when executed by a processor, implements the above disease prediction method, such as steps S1 to S4 in FIG. 1. The computer readable storage medium 200 includes, but is not limited to RAM, ROM, EEPROM, flash memory or other memory means, CD-ROM, digital versatile disk (DVD) or other optical disk storage, magnetic cartridges, magnetic tapes, disk storage or other magnetic storage devices, or any other medium that can be used to store the desired information and can be accessed by a computer. In addition, it is well known to those of ordinary skill in the art that communication media typically contain computer readable instructions, data structures, program modules, or other data in modulated data signals such as carrier waves or other transmission mechanisms, and may include any information delivery medium.

It will be understood that the above embodiments and implementations are only exemplary embodiments adopted to illustrate the principles of the present disclosure, but the present disclosure is not limited thereto. For a person of ordinary skill in the art, various improvements and variations can be made without departing from the spirit and substance of the present disclosure, and these variations and improvements are also considered to be within the scope of protection of the present disclosure.

1. A disease prediction method, comprising:
acquiring gene sequencing data of a test sample;
performing data analysis on the gene sequencing data to obtain a mutation site in the gene sequencing data;
performing a mutation annotation of the mutation site to obtain mutation-related information of the mutation site;
predicting a score of influence degree of the mutation site on gene function based on the mutation-related information of the mutation site; and
annotating the mutation site with a disease based on the score of influence degree of the mutation site on gene function and a predefined disease database to obtain a mitochondrial disease corresponding to the mutation site;
wherein, when a first mitochondrial disease corresponding to the mutation-related information of the mutation site is recorded in the predefined disease database, the first mitochondrial disease is taken as the mitochondrial disease corresponding to the mutation site; when the first mitochondrial disease is not recorded in the predefined disease database and the score of influence degree is greater than a predetermined threshold, a second mitochondrial disease corresponding to an adjacent site of the mutation site is acquired from the predefined disease database, and the second mitochondrial disease is taken as the mitochondrial disease corresponding to the mutation site.

2. The disease prediction method according to claim 1, wherein the mutation-related information comprises: a mutation type, a mutation region, a mutation location, and a mutation-induced variation in a CDS and a protein.

3. The disease prediction method according to claim 2, wherein predicting the score of influence degree of the mutation site on gene function based on the mutation-related information of the mutation site comprises:
separately predicting scores of influence degree of different mutation-related information on gene function to obtain multifaceted scores of influence degree, and
determining the score of influence degree of the mutation site on the gene function based on the multifaceted scores of influence degree.

4. The disease prediction method according to claim 3, wherein separately predicting the scores of influence degree of different mutation-related information on gene function to obtain the multifaceted scores of influence degree comprises:
acquiring, as a first score, a score of influence degree of the mutation site on conservativeness and physicochemical properties of a protein;
acquiring, as a second score, a score of influence degree of the mutation type of the mutation site on the gene function; and
acquiring, as a third score, a score of influence degree of the mutation location of the mutation site on the gene function;
wherein the multifaceted scores of influence degree comprise: the first score, the second score and the third score;
determining the score of influence degree of the mutation site on the gene function based on the multifaceted scores of influence degree specifically comprises: determining the score of influence degree of the mutation site on the gene function according to the following formula,
$$S = λ1 * Si + λ2 * St + λ3 * Sp ,$$
wherein S is the score of influence degree of the mutation site on the gene function, Si is the first score, St is the second score, Sp is the third score; λ1, λ2 and λ3 are predetermined weights, and λ1+λ2+λ3=1.

5. The disease prediction method according to claim 4, wherein λ1 and λ2 each range from 0.15 to 0.25, and λ3 ranges from 0.5 to 0.7.

6. The disease prediction method according to claim 4, wherein acquiring the score of influence degree of the mutation site on the conservativeness and physicochemical properties of the protein comprises:
analyzing the mutation-related information of the mutation site separately by a plurality of prediction tools to predict a plurality of reference scores of influence degree of the mutation site on the conservativeness and physicochemical properties of the protein, and
averaging the plurality of reference scores of influence degree as the first score.

7. The disease prediction method according to claim 4, wherein acquiring the score of influence degree of the mutation type of the mutation site on the gene function comprises:
determining, based on a predetermined first mapping relationship, the score of influence degree of the mutation type of the mutation site on the gene function; wherein scores of influence degree of a plurality of different mutation types on gene function are recorded in the first mapping relationship.

8. The disease prediction method according to claim 4, wherein the mutation location comprises a location number n of the mutation site in a protein sequence; and
acquiring the score of influence degree of the mutation location of the mutation site on gene function specifically comprises:
determining the third score according to the following formula, when the mutation type of the mutation site is a drift mutation or a nonsense mutation,
Sp=1−n/L, where L is the length of the protein sequence, or
determining the third score as 0 when the mutation type of the mutation site is any other than the drift mutation and the nonsense mutation.

9. The disease prediction method according to claim 1, wherein acquiring the gene sequencing data of a test sample comprises:
acquiring initial gene sequencing data of the test sample, and
filtering the initial gene sequencing data to obtain the gene sequencing data.

10. The disease prediction method according to claim 9, wherein acquiring the initial gene sequencing data of the test sample comprises:
acquiring the initial gene sequencing data of the test sample using Nanopore sequencing technology or targeted enrichment sequencing technology.

11. The disease prediction method according to claim 1, wherein performing data analysis on the gene sequencing data to obtain the mutation site in the gene sequencing data comprises:
comparing the gene sequencing data with a reference sequence of a reference mitochondrial genome, and determining comparison result data, the comparison result data comprising a site of the gene sequencing data in the reference mitochondrial genome,
performing a mutation detection on the comparison result data to determine the mutation site in the comparison result data.

12. The disease prediction method according to claim 11, wherein performing a mutation detection on the comparison result data to determine the mutation site in the comparison result data comprises:
performing a SNV detection on the comparison result data to obtain a first detection result, the first detection result comprising a SNV site included in the comparison result data; and
performing an indel detection on the comparison result data to obtain a second detection result, the second detection result comprising an indel site included in the comparison result data;
wherein the mutation site comprises the SNV site and the indel site.

13. A disease prediction device, comprising:
a data acquisition module configured to acquire gene sequencing data of a test sample;
an analysis module configured to perform data analysis on the gene sequencing data to obtain a mutation site in the gene sequencing data;
a mutation annotation module configured to perform mutation annotation of the mutation site to obtain mutation-related information of the mutation site;
a prediction module configured to predict a score of influence degree of the mutation site on gene function based on the mutation-related information of the mutation site; and
a disease annotation module configured to annotate the mutation site with a disease based on the score of influence degree of the mutation site and a predefined disease database to obtain a mitochondrial disease corresponding to the mutation site;
wherein, when a first mitochondrial disease corresponding to the mutation-related information of the mutation site is recorded in the predefined disease database, the first mitochondrial disease is taken as the mitochondrial disease corresponding to the mutation site; when the first mitochondrial disease is not recorded in the predefined disease database and the score of influence degree is greater than a predetermined threshold, a second mitochondrial disease corresponding to an adjacent site of the mutation site is acquired from the predefined disease database, and the second mitochondrial disease is taken as the mitochondrial disease corresponding to the mutation site.

14. An electronic device comprising a memory and a processor, the memory having a computer program stored thereon, wherein the computer program, when executed by the processor, implements the method of claim 1.

15. A computer readable storage medium having a computer program stored thereon, wherein the computer program, when executed by a processor, implements the method of claim 1.

16. The disease prediction method according to claim 4, wherein the second score is 0 when the mutation type of the mutation site is the same mutation or intergenic region mutation, or
the second score is 0.5 when the mutation type of the mutation site is nonsynonymous mutation or non-drift mutation; or
the second score is 1 when the mutation type of the mutation site is nonsense mutation or drift mutation.

17. The disease prediction method according to claim 6, wherein the multiple prediction tools are selected from PANTHER, PolyPhen-2 and SIFT, each predicting the influence degree of the mutation site on conservativeness and physicochemical properties of the protein, wherein the influence degree of the mutation site on conservativeness and physicochemical properties of the protein is one of the following four outcomes: no influence, possible influence, deleterious, and unable to predict.

18. The disease prediction method according to claim 17, wherein when the mutation site has no influence on the conservativeness and physicochemical properties of the protein, a reference score of influence degree output by the prediction tool is 0; when the mutation site has possible influence on the conservativeness and physicochemical properties of the protein, the reference score of influence degree output by the prediction tool is 0.5; when the mutation site has a deleterious influence on the conservativeness and physicochemical properties of the protein, the reference score of influence degree output by the prediction tool is 1; and when the influence degree of the mutation site on the conservativeness and physicochemical properties of the protein is not predictable, the reference score of influence degree output by the prediction tool is no score.

19. The disease prediction method according to claim 17, wherein a reference score of influence degree obtained from PANTHER is denoted as SPANTHER, the reference score of influence degree obtained from PolyPhen-2 is denoted as SPolyPhen-2, and the reference score of influence degree obtained from SIFT is denoted as SSIFT, the first score Si=(SPANTHER+SPolyPhen-2+SSIFT)/N, wherein N is the number of prediction tools giving scores.

20. The disease prediction method according to claim 12, wherein the SNV detection is performed using a longshot tool.
